(19) United States
(12) Patent Application Publication
(10) Pub. No.: US 2012/0231448 A1
(43) Pub. Date: Sep. 13, 2012

(54) LOW CELL TOXICITY ANTIBIOTIC HYGROMYCIN B

(75) Inventor: Qiubao PAN, Singapore (SG)

(73) Assignee: TOKU-E Company, Bellingham, WA (US)

(21) Appl. No.: 13/414,056

(22) Filed: Mar. 7, 2012

Related U.S. Application Data
(60) Provisional application No. 61/450,490, filed on Mar. 8, 2011.

(51) Int. Cl. C12Q 1/68; C12Q 1/04; C12N 5/071; C07H 15/224; C07H 1/06

(52) U.S. Cl. 435/6.1; 536/16.8; 536/16.9; 435/373; 435/34

(57) ABSTRACT

A preparation of antibiotic hygromycin B with low cell toxicity and high purity, and methods of preparing such a preparation, are provided. More specifically, an isolated antibiotic hygromycin B with a purity of greater than 98% and impurities C, D and E individually less than 0.5% and impurity F less than 2%, as measured by HPLC, is described. Uses of this high purity antibiotic hygromycin B include, for example, for in vitro cell selection.

### CROSS REFERENCE TO RELATED APPLICATION

This application claims the benefit under 35 U.S.C. §119(e) of U.S. Provisional Patent Application No. 61/450,490 filed Mar. 8, 2011, which application is incorporated herein by reference in its entirety.

### BACKGROUND

1. Technical Field

The present invention relates generally to a preparation of antibiotic hygromycin B with low cell toxicity and high purity, and methods of producing such a preparation. More specifically, the present invention relates to an isolated antibiotic hygromycin B with a purity of greater than 98% and impurities C, D and E individually less than 0.5% and impurity F less than 2%, as measured by HPLC.

2. Description of the Related Art

The antibiotic known as “hygromycin B” is an aminoglycoside antibiotic and thus belongs to the class of antibiotics that includes gentamicin, neomycin, paromycin, sisomicin and the like. Hygromycin B possesses one secondary and two primary amino groups, of which one or more of the amino groups when charged may form salts, such as acid addition salts. Typically, hygromycin B is in the form of a mono- or di-salt. Common forms of hygromycin B are a monohydrochloride salt or a dihydrochloride salt. These hydrochloride salt forms may be generated by exposing hygromycin B base to hydrochloric acid to convert the base form to a corresponding acid addition salt at one or more of the primary amino group positions on hygromycin B.

The biological activity of antibiotic hygromycin B is to inhibit polypeptide synthesis in both prokaryotic and eukaryotic cells. Hygromycin B is normally used in animal feed to free infected animals from animal parasites such as worms. It is also normally used in laboratory research, for example, to select genetically engineered cells. Hygromycin B is commonly used for the selection of prokaryotic and eukaryotic cells stably transfected with hygromycin resistance genes; and for the maintenance of the phenotype of hygromycin resistant cells. Resistance to hygromycin B is conferred by a resistance gene which encodes a kinase that inactivates hygromycin B through phosphorylation. Examples of sources of prokaryotic cells are bacteria. Examples of sources of eukaryotic cells are mammals, insects, algae, yeast, fungi, amoebae and plants. An example of an amoeba is dictyostelium. Examples of cells from mammals are cells from human and non-human mammals, such as rodents and non-human primates. Examples of sources of rodent cells are mice, rats and hamsters.

Hygromycin B is commonly used by researchers desiring a selection agent in gene transfer experiments. Antibiotic-resistant genes are used as markers to identify the successful transfer of other genes. Transfection of the hygromycin resistance genes into cells produces resistance to hygromycin B and allows the cells to grow in media containing hygromycin B. In addition to its use in gene transfer experiments, hygromycin B may be used for the elimination of cells (such as fibroblasts) contaminating mixed cell cultures.

Hygromycin B is produced in nature by a species of the microorganism Streptomyces. The species is S. hygroscopicus. The preliminary characterization of hygromycin B was described by Mann and Bromer (J. Am. Chem. Soc. 80:2714-2716, 1958). Production of partially purified hygromycin B is disclosed in U.S. Pat. No. 3,018,220 (the '220 Patent), naming McGuire and Mann as inventors. The chemical structure of hygromycin B and the biochemical basis of the resistance to hygromycin B in S. hygroscopicus, the producing organism, is described in Pardo et al. (J. Gen. Microbiol. 131:1289-1298, 1985).

The processes disclosed in the '220 Patent for producing a hygromycin B preparation are the commonly used processes in the art. Example 1 of the '220 Patent describes initial isolation of hygromycin B from fermentation broth obtained from tank fermentation of Streptomyces hygroscopicus. The initial isolation of hygromycin B involves the use of Amberlite IRC-50 cation exchange resin (“IRC-50”). In the '220 Patent, the eluate of the ion exchange resin that contains crude hygromycin B is subjected to exposure to activated charcoal.

The potency of killing hygromycin B-sensitive cells is not the same for all preparations of partially purified hygromycin B. Therefore, if a preparation of hygromycin B contains other hygromycins or hygromycin-like antibiotics, the cell-killing potency will be different than that for high purity antibiotic hygromycin B. The problem is made worse, for example, due to the level of impurities in general varying, or the ratio of impurities relative to each other varying, from batch to batch of hygromycin B.

In order for the selection of sensitive versus resistant cells to work, the cytotoxicity of the hygromycin B plus its impurities (which possess different cell-killing potencies) has to be very different for sensitive cells and resistant cells. For each batch of hygromycin B with varying impurity profiles (e.g., type, level or ratio, or combinations thereof), a researchist must re-optimize the concentration of hygromycin B containing impurities so that at least 90% of the sensitive cells are killed and at least 90% of the resistant cells survive without much damage. The re-optimization process requires expenditures of time and resources. Further, the presence of certain impurities in commercially available hygromycin B causes the loss of resistant cells, unless low concentrations are used which then significantly increases the time to kill the sensitive cells.

Accordingly, there is a need in the art of in vitro cell selection for an improved preparation of antibiotic hygromycin B that is of higher purity than the preparations currently commercially available. The present invention fulfills this need and further provides other related advantages.

### BRIEF SUMMARY

Briefly stated, a preparation of antibiotic hygromycin B with low cell toxicity and high purity, and methods of producing such a preparation, are provided. This highly pure antibiotic hygromycin B with low cell toxicity may be used for a variety of purposes including, for example, for in vitro cell selection.

In an embodiment, the present invention provides an isolated antibiotic hygromycin B with low cell toxicity having a purity of greater than 98% and impurities C, D and E individually less than 0.5% and impurity F less than 2%, wherein the purity and the impurities are measured by high performance liquid chromatography (HPLC), and hygromycin B having formula I:

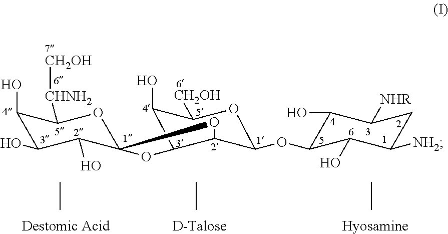

where R of formula I is CH₃ in hygromycin B;

wherein impurity F having formula I where R is H;

wherein impurity D having formula II:

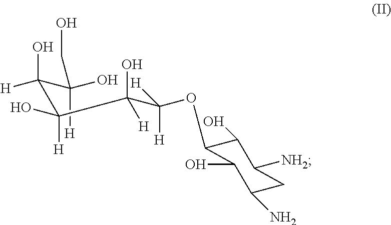

and wherein impurities C and E having formula III:

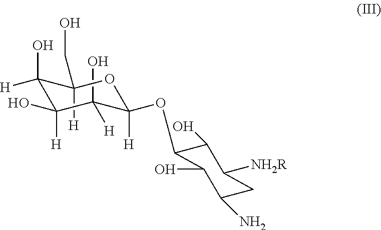

where R of formula III is H in impurity C, and R of formula III is CH₃ in impurity E.

In an embodiment, the isolated antibiotic hygromycin B is in combination with a carrier or diluent acceptable to one or more cell type.

In an embodiment, the present invention provides a method of producing antibiotic hygromycin B with low cell toxicity having a purity of greater than 98% and impurities C, D and E individually less than 0.5% and impurity F less than 2%, wherein the purity and the impurities are measured by HPLC, and hygromycin B and impurities C, D, E and F having the formulae set forth herein, comprising the steps of:

(a) dissolving hygromycin B, having purity of 83% up to 98% by HPLC, in sufficient water to make a 50% (w/v) solution;

(b) exposing the solution of step (a) to silica gel loaded into a column which is maintained at 20° C., wherein the silica gel has been balanced with standard pH 8 phosphoric buffer;

(c) contacting the silica gel of step (b) with additional standard pH 8 phosphoric buffer to elute destomic acid (6.9 minutes), impurity C (6.9 minutes) and impurity D (7.1 minutes);

(d) contacting the silica gel of step (c) with standard pH 8 phosphoric buffer at three times the standard concentration at a flow rate of 25 ml/minute to elute impurity E and impurity F;

(e) repeating step (d) for ten bed volumes, or greater if monitoring for impurities by HPLC warrants; and

(f) contacting the silica gel of step (e) with 0.05 M HCl to desorb hygromycin B, thereby yielding hygromycin B with a purity of greater than 98% as measured by HPLC.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 depicts the chemical structure of hygromycin B where R is CH₃; and the chemical structure of impurity F where R is H.

FIG. 2 depicts the chemical structure of impurity D.

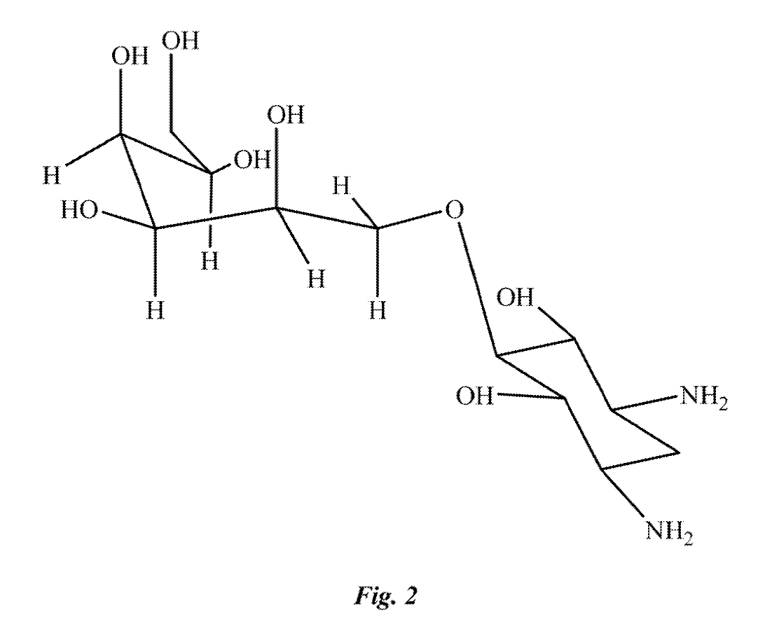

FIG. 3 depicts the chemical structure of impurity C where R is H, and impurity E where R is CH₃.

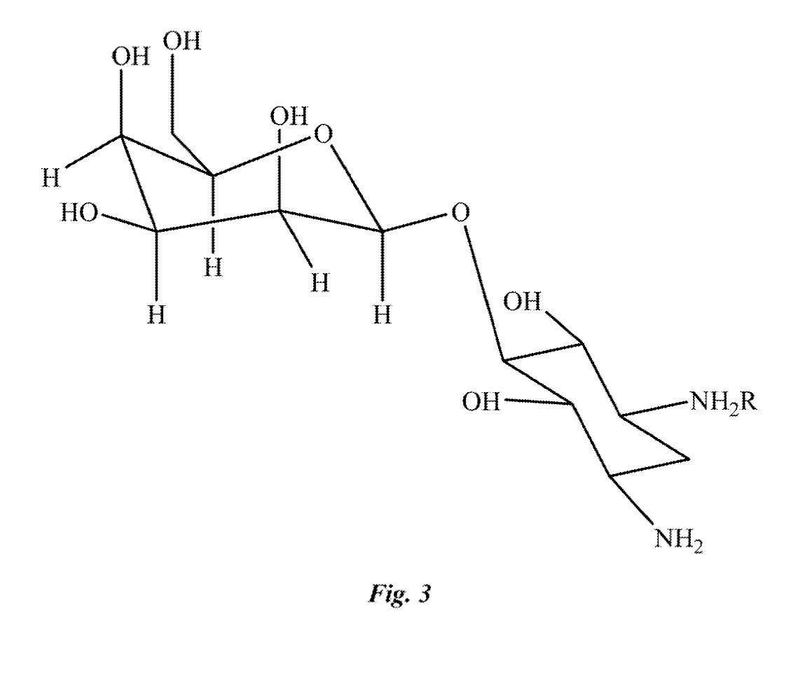

FIG. 4 depicts the chemical structure of destomic acid, which is a component of hygromycin B.

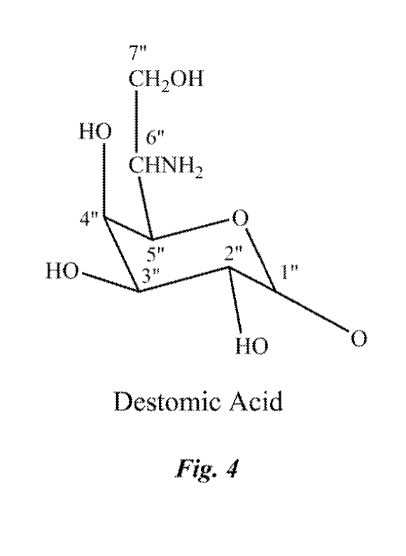

FIG. 5 is a chromatogram from HPLC of commercial grade hygromycin B (Invitrogen, Carlsbad, Calif.). The chromatogram shows the amount of hygromycin B relative to impurities in the commercial grade hygromycin B preparation.

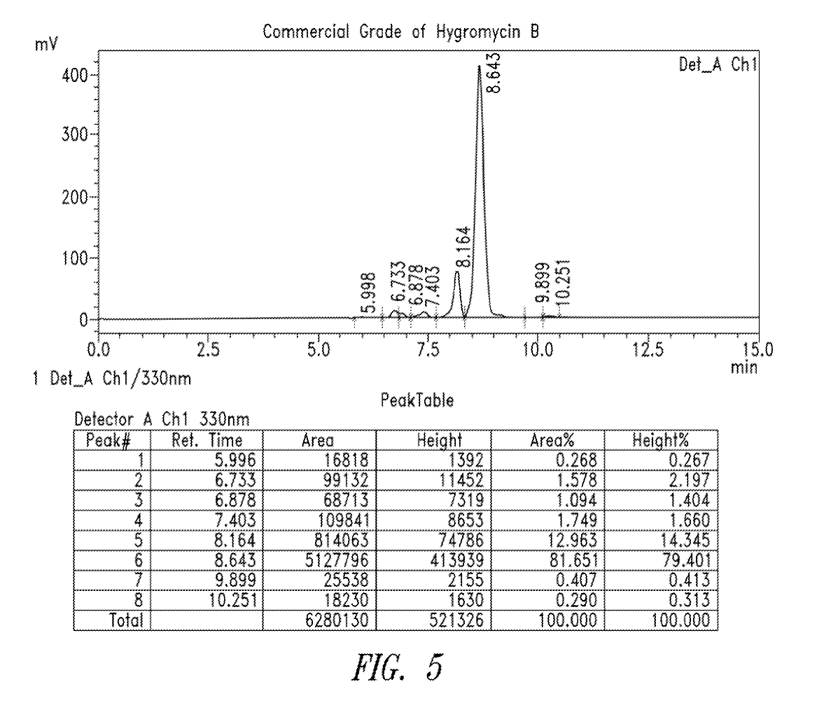

FIG. 6 is a chromatogram from HPLC of high purity grade hygromycin B of the present invention (prepared according to the methods disclosed herein). HPLC was performed in a manner identical to that of the HPLC used to produce the chromatogram of FIG. 5.

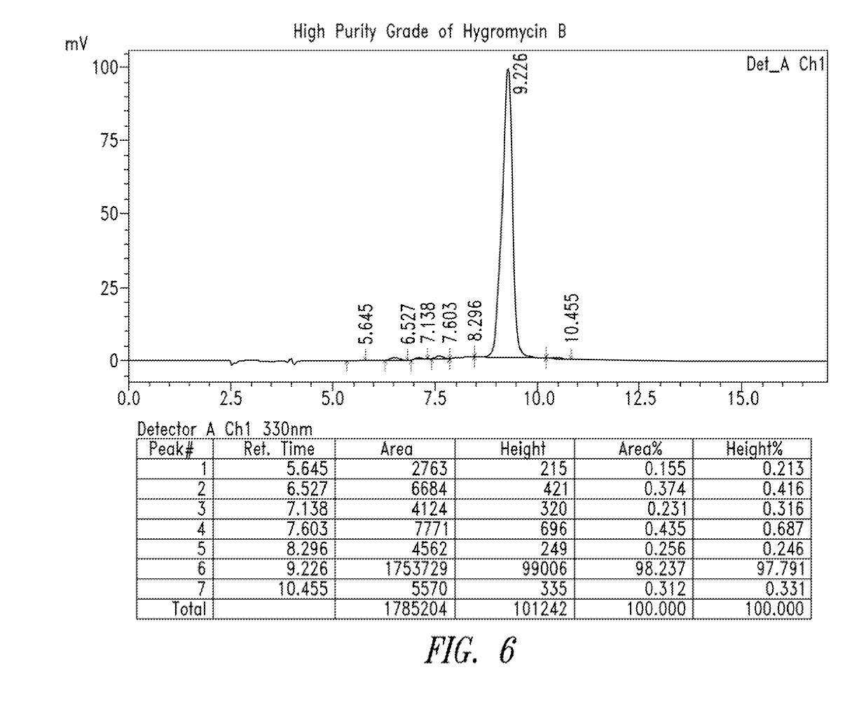

### DETAILED DESCRIPTION

As noted above, the present invention provides a preparation of antibiotic hygromycin B with low cell toxicity and high purity, and methods of producing such a preparation. This highly pure antibiotic hygromycin B with low cell toxicity has a variety of in vitro uses.

As used herein, the term “antibiotic hygromycin B” (also referred to simply as “hygromycin B” herein) includes salts thereof, such as acid addition salts. For example, monosulfate and disulfate salts of hygromycin B are included. The non-salt form of hygromycin B is “hygromycin B base” and is where all primary amino groups of hygromycin B are unprotonated. Sulfate salts of hygromycin B may be prepared by reaction of hygromycin B base with sulfuric acid. Alternatively, sulfuric acid may be substituted with an equivalent of other inorganic or organic acids to form salts other than sulfate salts. As used herein, the phrase “low cell toxicity” refers to low toxicity to cells resistant to hygromycin B.

The percent purity of antibiotic hygromycin B and the percent impurity of individual impurities (for example, impurities C, D, E and F) within a hygromycin B preparation may be measured by high performance liquid chromatography (herein referred to as “HPLC”). The specific protocol used herein to assay hygromycin B purity and impurities is that described by Kubo et al. in J. of Chromotography 227:244-248, 1982, which disclosure is specifically incorporated by reference rather than reproduced herein since it is part of the art and well known to those in the art. As used herein, the phrases “wherein the purity and the impurities are measured by high performance liquid chromatography” or “wherein the purity and the impurities are measured by HPLC” or “as measured by high performance liquid chromatography” or “as measured by HPLC” or “using HPLC” or the like, each refer to the HPLC methodology described by the Kubo et al. journal article (as cited above).

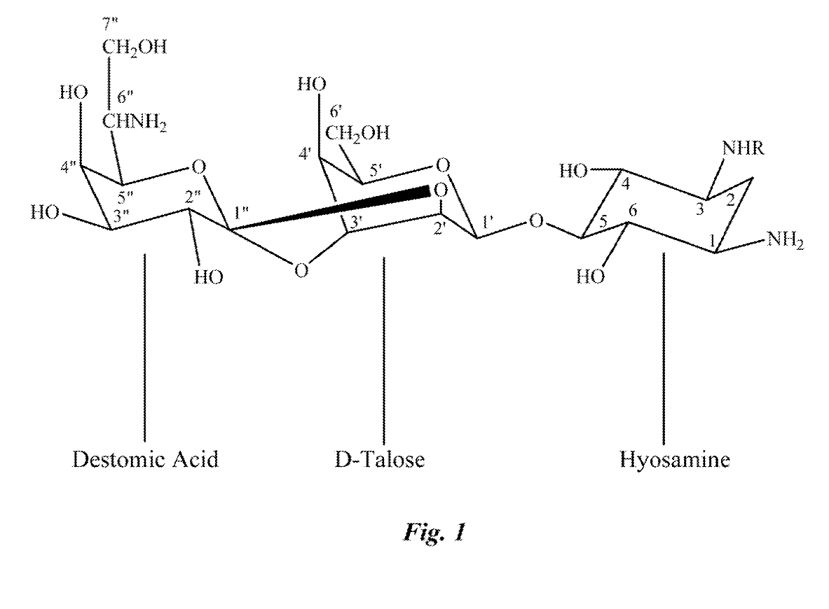

Many manufacturers of antibiotic hygromycin B use the processes of making hygromycin B that are disclosed in U.S. Pat. No. 3,018,220 (the '220 Patent) and the journal article of Mann and Bromer, J. Am. Chem. Soc. 80:2714-2716 (1958), which are described in the BACKGROUND section herein. When a commercial grade hygromycin B was tested in the context of the present invention using HPLC, the commercial grade of hygromycin B had only a maximum of 90% purity (FIG. 1). Some of the commercial grade hygromycin B have over 10% impurities. In the commercial grade of hygromycin B, there are hygromycin B-like impurities, including impurity F as described herein. Relative to hygromycin B, most of the impurities have reduced or no antibiotic activity relevant to, for example, cell selection. Thus, the potency of killing sensitive versus resistant cell lines is not the same among the impurities. Since the levels of impurities present can vary relative to hygromycin B, and the ratio of particular impurities can vary among themselves, from batch to batch of commercial grade hygromycin B, the concentration of a particular hygromycin B preparation needed to effect about 90% killing of sensitive cells with 90% survival of resistant cells will vary relative to other hygromycin B preparations.

The present invention provides isolated antibiotic hygromycin B with low cell toxicity and high purity. The purity is greater than 98% and impurities C, D and E individually less than 0.5% and impurity F less than 2%, as measured by HPLC. This is a significantly greater purity than most commercially available preparations and better purity than even the best commercial grade hygromycin B currently available. This antibiotic hygromycin B of the present invention having high purity and low cell toxicity is an unexpected improvement in the art.

In the present invention, the production of antibiotic hygromycin B with low cell toxicity having high purity is effected (as described in detail in the EXAMPLES section herein) by the use of silica gel in combination with the use of certain elution solvents with particular concentrations which are specific to loading of a partially purified preparation and elution of major impurities versus elution of hygromycin B from the silica gel preparation.

The isolated antibiotic hygromycin B of the present invention with low cell toxicity having a purity of greater than 98% and impurities C, D and E individually less than 0.5% and impurity F less than 2%, as measured by HPLC, has a variety of in vitro uses. One of the uses is for cell selection. For example, this high purity hygromycin B may be used for selection and maintenance of prokaryotic and eukaryotic cells stably transfected with neomycin resistance genes. This methodology is commonly used in vitro in gene transfer experiments. Another use of this high purity hygromycin B may be for the elimination of cells contaminating mixed cell cultures. An example of cells that may be desired to be eliminated are fibroblasts. The above uses and others are well known to those in the art.

The following Examples are offered by way of illustration and not by way of limitations.

### EXAMPLES

### Example 1

### Testing of Different Batches of Commercially Available Hygromycin B

For comparison purposes, the main HPLC impurities (over 1%) in hygromycin B batches from different suppliers are listed in Table 1 by HPLC retention time. The minor impurities (less than 1%) are not included in Table 1, except for T-99. T-99 (over 99%) is prepared by Preparative HPLC with silica gel as the stationary phase.

TABLE 1
HPLC MAJOR IMPURITIES (OVER 1%) FOR DIFFERENT
BATCHES OF HYGROMYCIN B IDENTIFIED AS DIFFERENT
RETENTION TIMES

| HPLC | Batch of Hygromycin B | | | | | Impurity |
|---|---|---|---|---|---|---|
| Retention | S128 | I-A10 | R-01 | T-94 | T-99 | assigned name or |
| Time | (%) | (%) | (%) | (%) | (%) | Hygromycin B |
| 6.0 min | 2.17 | — | 2.27 | — | — | A |
| 6.9 min | 5.11 | 1.0 | 5.97 | — | — | C |
| 7.1 min | 1.59 | — | — | — | — | D |
| 7.4 min | 6.54 | 1.75 | 5.92 | 1.2 | 0.5 | E |
| 8.1 min | 18.5 | 14.35 | 15.4 | 1.55 | | F |
| 8.6 min | 64.9 | 79.4 | 66.7 | 94.7 | 99.0 | Hygromycin B |

S128 is from Sigma (St. Louis, MO);
I-A10 is from Invitrogen (Carlsbad, CA);
R-01 is from Ameresco (Solon, OH);
T-94 is produced by TOKU-E (Bellingham, WA); and
T-99 by the methods described herein.

From the above impurities profile table (Table 1), it can be seen that the main impurities are very similar for 5128, I-A 10 and R-01, except the amount of impurities may be different. The five major impurities are designated as impurity A (6.0 min retention time), impurity C (6.9 min), impurity D (7.1 min), impurity E (7.4 min) and Impurity F (8.1 min). Impurity A is identified as destomic acid. Impurity E is identified as hydrolysis of hygromycin B.

TABLE 2
LD 50 HYGROMYCIN B CONCENTRATION (μG/ML) FOR
SENSITIVE CELLS FROM DIFFERENT COMMERCIAL SOURCES

| Sensitive | Batch | | | | |
|---|---|---|---|---|---|
| Cells | S128 | I-A10 | R-01 | T-94 | T-99 |
| Tobacco | 4.3 | 4.5 | 4.1 | 4.7 | 5.2 |
| Calli cells | | | | | |
| 293 cell | 8.8 | 8.3 | 9.2 | 8.5 | 8.1 |
| Fungi cell | 13.2 | 14.6 | 13.7 | 14.2 | 13.8 |
| Escherichia | 8.2 | 8.1 | 8.4 | 8.2 | 8.6 |
| Coli | | | | | |

From the above table (Table 2), it can be seen that the Lethal Dose 50 (LD 50) hygromycin B concentration for sensitive cells is about the same for these suppliers of hygromycin B.

TABLE 3
LD 50 HYGROMYCIN B CONCENTRATION (μG/ML) FOR
RESISTANT CELLS FROM DIFFERENT VENDORS

| | Hygromycin B Batches | | | | |
|---|---|---|---|---|---|
| Resistant Cells | S128 | I-A10 | R01 | T94 | T99 |
| Tobacco Calli cells transformed | 72 | 220 | 81 | 490 | 720 |
| with the Hygromycin B | | | | | |
| phosphotransferase gene | | | | | |
| 293 cells transformed | 51 | 136 | 69 | 394 | 570 |
| with the Hygromycin B | | | | | |
| phosphotransferase gene | | | | | |

From Table 3, it can be seen that T-94 (94%) and T-99 (99%) hygromycin B have lower toxicity to resistant cells relative to S 128, I-A10 and R01. It is possible that the toxicity to the resistant cells is mainly due to the 7.1 minute HPLC retention time impurity (impurity D). T-99 prepared by the present invention has much lower toxicity to the resistant cells. Hygromycin B from Sigma Chemicals (S128) and Ameresco (R01) have much worse selectivity compared to both TOKU-E′s product (T-94) and Invitrogen's product (I-A10).

### LD 50 Comparison for Resistant Cells for Different Cell Lines:

The impurities of commercial hygromycin B are collected during the preparation of the high purity hygromycin B. Each impurity is individually isolated by preparative HPLC with C18 as the stationary phase for preparation of hygromycin B with HPLC purity greater than 99%. The impurities are then used to add to the media of the following cell line to test the toxicity of the individual impurities to cell lines carrying the resistant gene of hygromycin B.

TABLE 4
LD 50 FOR DIFFERENT IMPURITIES (μG/ML) FOR
RESISTANT CELLS

| | Impurities | | | | |
|---|---|---|---|---|---|
| Resistant Cells | A | C | D | E | F |
| Tobacco Calli cells | 193 | 2.9 | 0.5 | 4.2 | 16 |
| transformed with the | | | | | |
| Hygromycin B | | | | | |
| phosphotransferase gene | | | | | |
| 293 cells transformed | 240 | 7.2 | 1.7 | 3.2 | 11 |
| with the Hygromycin B | | | | | |
| phosphotransferase gene | | | | | |

From Table 4, it can be seen that the toxicity of different impurities varies for different cell lines and the toxicity is much higher than the purified hygromycin B of the present invention (comparing toxicity of impurities versus toxicity of 500-700 μg/ml of T-99). The impurities may not share the same resistant gene as hygromycin B itself. In order to make low cell toxicity hygromycin B, all the impurities need to be controlled. In particular, the concentrations of impurities C, D and E are relatively more important to the toxicity to the resistant cells.

Table 5 is a killing curve comparison between hygromycin B of the present invention (T-99) and Invitrogen's hygromycin B (I-A10).

TABLE 5
DEATH RATIO OF DIFFERENT RESISTANT CELLS FOR
SEVERAL HYGROMYCIN B CONCENTRATIONS OF
HYGROMYCIN B BATCH T-99 AND I-A10

| Hygromycin B | T-99 + | T-99 + | I-A10 + | I-A10 + |
|---|---|---|---|---|
| concentration | Tobacco cells | 293 cells | Tobacco cells | 293 cells |
| 0.1 μg/ml | 100 | 100 | 100 | 100 |
| 1 μg/ml | 99.7 | 100 | 99.2 | 98.6 |
| 10 μg/ml | 100 | 100 | 83.6 | 81.2 |
| 100 μg/ml | 97.3 | 90.4 | 65.2 | 51.5 |
| 1000 μg/ml | 43.6 | 39.5 | 23.7 | 16.3 |
| 3000 μg/ml | 11.7 | 50.7 | 1.8 | 0.0 |
| 5000 μg/ml | 6.2 | 1.5 | 0.0 | 0.0 |
| 10000 μg/ml | 2.2 | 0 | 0.0 | 0.0 |

Table 5 shows that T-99 hygromycin B has much lower toxicity to the resistant cell lines tested.

The high hygromycin B concentration of high purity hygromycin B of the present invention kills the sensitive cells quicker than the lower concentration of commercial grade hygromycin B, and the resistant cell survival rate is much higher. Thus, the selection process is done more quickly with hygromycin B of the present invention than with presently available commercial grade hygromycin B.

This reduction in time to complete cell selection that is provided by the present invention is of significant benefit to researchers in industry and academia. Thus, the surprisingly high purified hygromycin B preparation of the present invention is of assistance to the progress of research and medical developments.

### Example 2

### Process of Preparing the Over 99% Pure Hygromycin B

### Preparation by Silica Gel

Silica gel 60 (EMD Chemicals, Gibbstown, N.J.) is balanced with standard pH 8 Phosphoric Buffer and the silica gel loaded into a column 80 mm (ID)×400 mm (height). Hygromycin B of 83% purity is dissolved by HPLC in water to make it 50% solution (w/v). Onto the column is loaded 180 ml of the hygromycin B solution. Impurities C and A (6.9 min), and D (7.1 min) are eluted with standard pH 8 phosphoric buffer. The other two impurities (E and F) are elated with 3 times concentration (3×) of standard pH 8 phosphoric buffer at a flow rate of 25 ml/min. The column temperature is maintained at 20° C. By elution of impurities with about 10 bed volume of 3× standard pH 8 phosphoric buffer, high purity hygromycin B can be eluted with 0.05M HCl. If desired, the chloride can be removed by anion ion exchange resin. The eluate can be concentrated, for example, by reverse osmosis. The elute can be vacuum dried.

### Example 3

### Structure of Hygromycin B and the Impurities of Hygromycin B

LC-MASS (liquid chromatography-mass spectroscopy) is used to identify the mass of the impurities isolated during the preparation of high purity hygromycin B. NMR is used to determine the basic chemical structures of the impurities. IR spectroscopy and MASS-MASS (tandem) spectroscopy are used to further confirm the structures. The structure of hygromycin B is as follows:

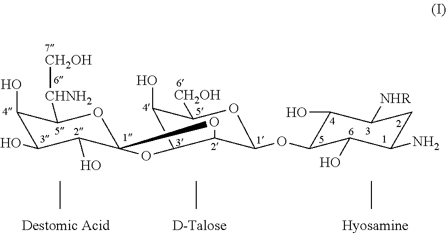

The above structure is hygromycin B where R═CH₃ and Impurity F where R═H.

All these impurities are hygromycin B related and the compositions of the impurities are as following:

| | Composition of impurities | MW |
|---|---|---|
| Impurity A | Destomic acid | 189.2 |
| Impurity C | D-Talose + 3-Demethyl-Hyosamine | 324.5 |
| Impurity D | Hydrolysis of Impurity C (D-Talose ring opens | 326.5 |
| | up) | |
| Impurity E | D-Talose + Hyosamine | 338.5 |
| Impurity F | Destomic acid + D-Talose + 3-Demethyl- | 513.5 |
| | Hyosamine | |
| Hygromycin B | Destomic acid + D-Talose + Hyosamine | 527.5 |

The following structure is Impurity C where R═H and Impurity E where R═CH₃.

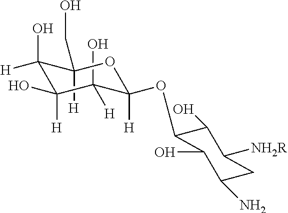

The following structure is Impurity A.

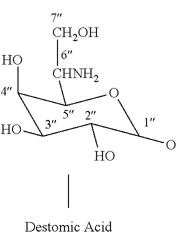

The following structure is Impurity D.

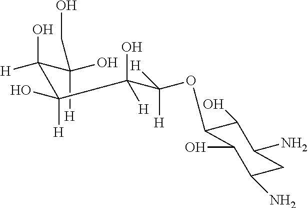

The toxicity is related to its structure. Pardo et al. (J. Gen. Microbiol. 131:1289-1298, 1985) found that the 7″-O-phosphorylation of the destomic acid ring in hygromycin B makes hygromycin B lose its biological activity both in vivo and in vitro. Impurities C, D and E have no destomic acid ring, but still have the hyosamine to maintain bioactivity.

### Example 4

### Method of Testing Hygromycin B by HPLC Using Post-Column Derivatization with O-Phthalaldehyde (OPA)

### (1). Preparation of Standard Solution

\textcircled{1}: Carefully weigh standard substance of hygromycin B, and dissolve with water to the concentration of 5 mg/ml.

\textcircled{2}: Preparation of the sample—Same as the preparation of standard substance.

### (2). Preparation of Mobile Phase Solution

Phase A
\textcircled{1}: Dissolve 26 g of sodium hydroxide in 1000 mL of water, and adjust with perchloric acid to a pH of 7.0
\textcircled{2}: Dissolve potassium dihydrogenphosphate (7.02 g), sodium 2,4-dimethylbenzenesulfonate (1.5 g) in the \textcircled{1} solution, then add 30 ml acetonitrile.

Phase B
\textcircled{1}: Dissolve 2.5 g of boracic acid in 100 mL of water, and adjust with 8N sodium hydroxide to a pH of 10.4 (boracic acid solution(0.4 mol/l) (pH 10.4).
\textcircled{2}: Dissolve 0.5 g of O-phthalaldehyde(OPA) in 3 mL of methanol, add 50 mL of boracic acid solution(0.4 mol/l) (pH 10.4), and 1 mL of mercaptoacetic acid, adjust with 8N sodium hydroxide to a final pH of 10.4, and add pure water to 900 ml, store in −4° C.
\textcircled{3}: Mix 0 with solution 0, and add 10 ml 4N potassium hydroxide before using.

### (3). Chromatograph System

The mobile phase A is for the column elution: 0.5 ml/min

The mobile phase B is for the derivation phase: 0.8 ml/min

Reaction temperature: 40° C.

Chromatographic column: C18, ODS, 250×4.6 mm SHIMADZHU

Column temperature: 40° C.

Injection vol.: 10 μl

Wavelength: 330 nm

Peak RT: 8-9 min

LC stop time: 15 min

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheetare incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An isolated antibiotic hygromycin B with low cell toxicity having a purity of greater than 98% and impurities C, D and E individually less than 0.5% and impurity F less than 2%, wherein the purity and the impurities are measured by high performance liquid chromatography (HPLC), and hygromycin B having formula I: 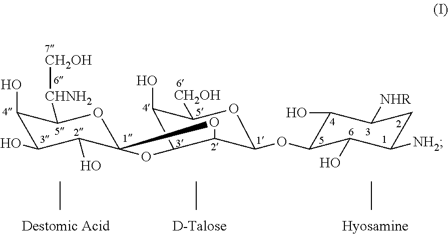
where R of formula I is CH₃ in hygromycin B;
wherein impurity F having formula I where R is H;
wherein impurity D having formula II: 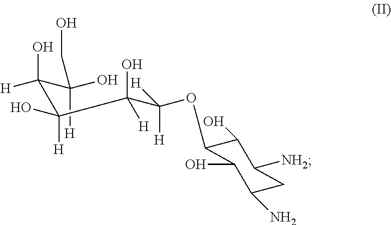
and
wherein impurities C and E having formula III: 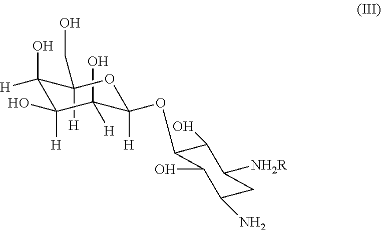
where R of formula III is H in impurity C, and R of formula III is CH₃ in impurity E.

2. The isolated antibiotic hygromycin B of claim 1 in combination with a carrier or diluent acceptable to one or more cell type.

3. A method of producing antibiotic hygromycin B with low cell toxicity having a purity of greater than 98% and impurities C, D and E individually less than 0.5% and impurity F less than 2%, wherein the purity and the impurities are measured by HPLC, and hygromycin B and impurities C, D, E and F having the formulae according to claim 1, comprising the steps of:
(a) dissolving hygromycin B, having purity of 83% up to 98% by HPLC, in sufficient water to make a 50% (w/v) solution;
(b) exposing the solution of step (a) to silica gel loaded into a column which is maintained at 20° C., wherein the silica gel has been balanced with standard pH 8 phosphoric buffer;
(c) contacting the silica gel of step (b) with additional standard pH 8 phosphoric buffer to elute destomic acid (6.9 minutes), impurity C (6.9 minutes) and impurity D (7.1 minutes);
(d) contacting the silica gel of step (c) with standard pH 8 phosphoric buffer at three times the standard concentration at a flow rate of 25 ml/minute to elute impurity E and impurity F;
(e) repeating step (d) for ten bed volumes, or greater if monitoring for impurities by HPLC warrants; and
(f) contacting the silica gel of step (e) with 0.05 M HCl to desorb hygromycin B, thereby yielding hygromycin B with a purity of greater than 98% as measured by HPLC.

4. The method of claim 3 with further step (g) comprising removing chloride from the hygromycin B of step (f) by anion ion exchange.

5. The method of claim 4 with further step (h) comprising converting the hygromycin B of step (g) to a dry state.
